(19) United States
(12) Patent Application Publication
(10) Pub. No.: US 2025/0271354 A1
(43) Pub. Date: Aug. 28, 2025

(54) SYSTEMS AND METHODS FOR OPERATING A SPECTROPHOTOMETER

(71) Applicant: ROCKLEY PHOTONICS LIMITED, Altrincham (GB)

(72) Inventors: Alexander FAST, Aliso Viejo, CA (US); Adam GARDNER, Mission Viejo, CA (US); Richard GROTE, Rancho Cucamonga, CA (US); Andrew George RICKMAN, Marlborough (GB); Casimir WIERZYNSKI, La Jolla, CA (US)

(21) Appl. No.: 19/064,480

(22) Filed: Feb. 26, 2025

Related U.S. Application Data
(60) Provisional application No. 63/558,611, filed on Feb. 27, 2024.

(51) Int. Cl. G01N 21/31; G01N 33/483; G06F 30/27

(52) U.S. Cl. CPC G01N 21/31; G01N 33/4833; G06F 30/27

(57) ABSTRACT

Systems and methods for operating a spectrophotometer. In some embodiments, a method includes: estimating the concentration of a biomarker in a tissue sample, the estimating including: performing a first simulation to calculate an optical path distribution for a tissue model; obtaining, with a spectrophotometer, a first absorbance spectrum based on the tissue sample; and estimating the concentration of the biomarker based on the optical paths and on the first absorbance spectrum.

### CROSS-REFERENCE TO RELATED APPLICATION(S)

The present application claims priority to and the benefit of U.S. Provisional Application No. 63/558,611, filed Feb. 27, 2024, entitled “SYSTEMS AND METHODS FOR OPERATING A SPECTROPHOTOMETER”, the entire content of which is incorporated herein by reference.

### FIELD

One or more aspects of embodiments according to the present disclosure relate to biomarker monitoring, and more particularly to systems and methods for operating a spectrophotometer.

### BACKGROUND

When monitoring, treating, or diagnosing a subject (e.g., a patient) it may be helpful to have information regarding the presence or concentration of various biomarkers (e.g., compounds such as glucose, creatinine, urea, lactate, or alcohol) within the tissues of the subject.

It is with respect to this general technical environment that aspects of the present disclosure are related.

### SUMMARY

According to an embodiment of the present disclosure, there is provided a method, including: estimating the concentration of a biomarker in a tissue sample, the estimating including: performing a first simulation to calculate an optical path distribution for a tissue model; obtaining, with a spectrophotometer, a first absorbance spectrum based on the tissue sample; and estimating the concentration of the biomarker based on the optical paths and on the first absorbance spectrum.

In some embodiments, the method includes performing a first plurality of simulations, including the first simulation, to calculate the optical path distribution.

In some embodiments, each of the simulations is a Monte-Carlo simulation.

In some embodiments, the method further includes: calculating an absorbance spectrum for each of the simulations; measuring a second absorbance spectrum of the tissue sample; and based on a similarity between: an absorbance spectrum for a second simulation of the simulations and the second absorbance spectrum, estimating a plurality of tissue physiology parameters of the tissue sample.

In some embodiments, the method further includes calculating a weight vector based on the tissue physiology parameters and on an absorption coefficient of the biomarker, wherein the estimating of the concentration of the biomarker includes calculating a dot product of the weight vector and the first absorbance spectrum.

In some embodiments, the method further includes: calculating an absorbance spectrum for each of the simulations; measuring a second absorbance spectrum of the tissue sample; and based on the similarity between: the absorbance spectrum for the second simulation and the second absorbance spectrum, estimating one or more parameters of the spectrophotometer.

In some embodiments, the method further includes calculating a weight vector based on the parameters of the spectrophotometer and on an absorption coefficient of the biomarker, wherein the estimating of the concentration of the biomarker includes calculating a dot product of the weight vector and the first absorbance spectrum.

In some embodiments, the method further includes: based on a similarity between: an absorbance spectrum for a second simulation of the simulations and the second absorbance spectrum, estimating a plurality of tissue physiology parameters of the tissue sample.

In some embodiments, the method further includes calculating a weight vector based: on the tissue physiology parameters, on parameters of the spectrophotometer, and on an absorption coefficient of the biomarker, wherein the estimating of the concentration of the biomarker includes calculating a dot product of the weight vector and the first absorbance spectrum.

### BRIEF DESCRIPTION OF THE DRAWINGS

These and other features and advantages of the present disclosure will be appreciated and understood with reference to the specification, claims, and appended drawings wherein:

FIG. 1 is a block diagram of a spectrophotometer, according to an embodiment of the present disclosure;

FIG. 2 is block diagram of a Monte Carlo model, according to an embodiment of the present disclosure;

FIG. 3 is a schematic drawing of the geometry of the interface between the spectrophotometer and the tissue of a subject, according to an embodiment of the present disclosure;

FIG. 4A is block diagram of a baseline simulation, according to an embodiment of the present disclosure; and

FIG. 4B is block diagram of an inverse Monte Carlo simulation, according to an embodiment of the present disclosure.

### DETAILED DESCRIPTION

The detailed description set forth below in connection with the appended drawings is intended as a description of exemplary embodiments of systems and methods for operating a spectrophotometer provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

In some embodiments, the skin of a subject may be illuminated by light form the output of a spectrophotometer; this light may scatter within the tissues of the subject, and a portion of the light may return to a photodiode of the spectrophotometer. The fraction of the light that returns to the photodiode, as a function of wavelength, may depend in part on the concentration of a biomarker of interest (e.g., glucose, or alcohol), which may have a characteristic absorption coefficient at each wavelength of the spectrophotometer. As such, it may be possible to infer to the concentration of a biomarker of interest from a measured absorbance spectrum. In some embodiments, the spectrophotometer is part of a wearable instrument, e.g., an instrument worn on the wrist of a subject.

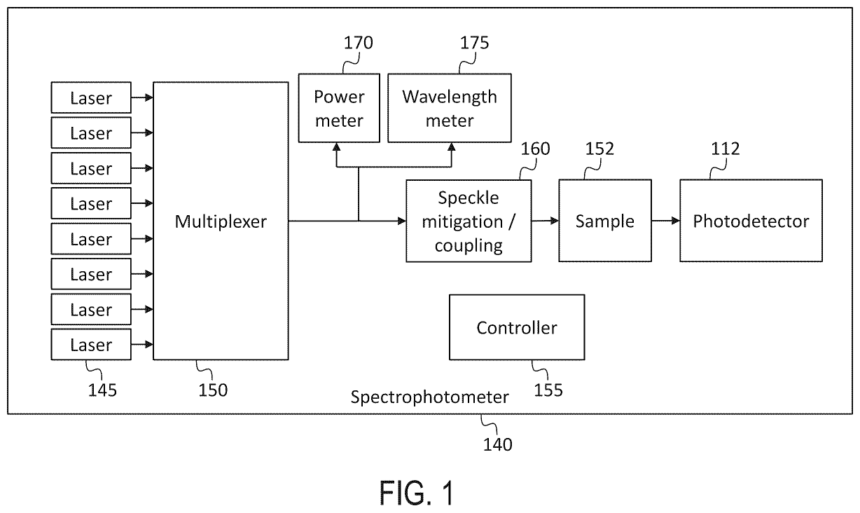

FIG. 1 is a block diagram of a spectrophotometer 140, in some embodiments. In some embodiments, as mentioned above, a spectrophotometer 140 (FIG. 1) may be used to measure various biomarkers, each of which may be the concentration of a constituent of the tissues of the subject. Each laser 145 of an array of lasers 145 (e.g., ten or more lasers 145, not all of which are shown) is connected to a wavelength multiplexer 150 (which may be, e.g., an arrayed waveguide grating, an echelle grating, or a cascade of Mach-Zehnder interferometers). Each laser 145 may include an InP reflective semiconductor optical amplifier (RSOA) coupled to a waveguide on a silicon photonic integrated circuit (a silicon PIC). The waveguide on the silicon photonic integrated circuit may include a grating reflector that sets the operating wavelength of the laser. Each laser 145 operates at a different respective wavelength and is connected to an input, corresponding to the operating wavelength of the laser, of the wavelength multiplexer 150. In operation, one laser is turned on at a time (e.g., by a controller 155, which may be or include a processing circuit), so that the combination of (i) the array of lasers 145 and (ii) the wavelength multiplexer 150 operates as a swept wavelength light source. In other embodiments, a different swept wavelength light source (e.g., a single widely tunable laser, or a source including an array of tunable lasers, each tunable over a different wavelength range) is used instead of the array of lasers 145 and the wavelength multiplexer 150 shown in FIG. 1. In the embodiment of FIG. 1, the wavelength separation between lasers 145 that are adjacent in wavelength may be between 5 nm and 50 nm, and the wavelength range may be about 2000 nm to 2500 nm (e.g., 2080 nm to 2400 nm). In some embodiments, one or more gaps may be present in the set of wavelengths (e.g., if a wavelength band within the range is of limited use because of strong absorption by water in the band).

Light from the output of the wavelength multiplexer 150 illuminates the sample 152. In some embodiments, a speckle mitigation system or coupling optics 160 for producing a beam of the desired shape in the sample 152, may be present between the output of the wavelength multiplexer 150 and the sample 152. After interacting with the sample in the sample 152, the light may be detected by the photodetector 112. In FIG. 1, the photodetector 112 is illustrated as being on the opposite side of the sample 152 from the source of the probe light for ease of illustration; in some embodiments the photodetector 112 is positioned on the same side of the sample as the source of the probe light, and the probe light may reach the photodetector 112 after scattering one or more times within the sample. This type of optical path may be important for measurements made by illuminating a first location on the skin of the subject with probe light (transmitted through a transmitting window), and detecting light returning from the skin at a second location near the first location (through a receiving window). The output radiance profile of the probe light may depend on the wavelength of the light and may affect the paths the light takes through the sample.

The photodiode signal may be amplified by a suitable amplifier, and converted to a digital signal by an analog to digital converter, and the resulting digital signal may be fed to the controller 155 for further processing. A power meter 170 and a wavelength meter 175 may measure the optical power and wavelength, respectively, of the probe light, and (i) corrections may be made (e.g., by the controller 155) by adjusting, e.g., the drive currents of the lasers or drive currents of heaters controlling the temperatures of respective gratings of the lasers, or (ii) errors in the transmitted power or wavelength may be compensated for when the data are analyzed. The ratio, as a function of wavelength, of (i) the optical power detected by the photodetector 112 to (ii) the optical power transmitted in the probe light may be referred to herein as an “absorbance spectrum”. Although the sample is shown illustrated within the boundaries of the spectrophotometer 140, it is not considered to be a part of the spectrophotometer.

The absorbance spectrum may depend, as mentioned above, on the concentration of the biomarker of interest within the tissue of the patient. The absorbance spectrum may also, however, depend on other factors, including, for example, (i) the various optical paths the light from the spectrophotometer may take in propagating through the tissue of the subject, and (ii) the absorption coefficient and relative abundance of each of a number of other components of the tissue of the subject. For example, if the absorbance spectrum is highly sensitive to a biomarker at each of a first wavelength and a second wavelength when there is little water in the tissue, and if water strongly absorbs the first wavelength but not the second wavelength, then the sensitivity of the absorbance spectrum to the biomarker may be reduced, by the presence of water, at the first wavelength but not (or less so) at the second wavelength.

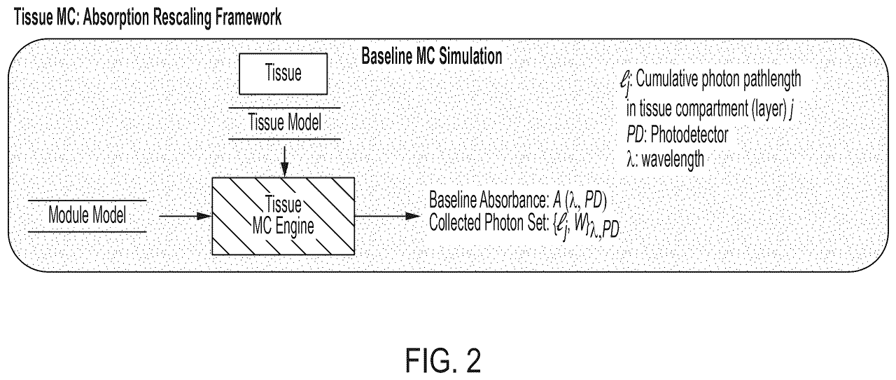

A suitable simulation (e.g., a Monte Carlo simulation, or a simulation using a different method for solving the radiative transfer equation) may be used to calculate the distribution of optical paths. FIG. 2 shows inputs and outputs of a Monte Carlo model. Each Monte Carlo simulation may include a plurality, or “ensemble” of iterations. Each iteration may include simulating (i) the launch of a ray of light (or “photon”) into the tissue of the subject, (ii) a plurality of scattering events, within the tissue of the subject, and (iii) the final detection or loss of the ray. The initial direction and position of each ray, which may correspond to an initial ray path within the tissue of the subject, may be selected randomly from the output radiance profile of the source. The probability density function used for this random selection may be proportional to the to the output radiance profile.

Each of the scattering events may be a transition between a current ray path and a subsequent ray path; for example the initial path from the transmitting aperture into the tissue of the subject may be a first ray path (a straight line segment in three-dimensional space); after a first scattering event the light may travel along a second ray path, and so forth. Eventually a ray path may satisfy a termination criterion and the iteration may end. Termination beyond threshold may be done using Russian Roulette which is an unbiased variance reduction approach.

The separation between the scattering events may be randomly selected using a pseudorandom number generated with a probability density function referred to herein as the scattering probability density function, which may be an exponential probability density function. At each scattering event the angle between an initial ray path and a scattered ray path (which may be the polar angle θ in a spherical polar coordinate system having the z-axis along the initial ray) may be randomly selected using a pseudorandom number generated with a probability density function referred to herein as the scattering phase function. The azimuthal angle (e.g., the azimuthal angle ¢ in the spherical polar coordinate system) may be randomly selected using a uniformly distributed pseudorandom number, under the assumption of random media. The scattering phase function may be, for example, a Henyey-Greenstein function (e.g., with a value of the g parameter that is within 15% of 0.8) or a Mie function.

Pseudorandom numbers with each of the various probability density functions used in embodiments disclosed herein may be generated from the output of a pseudorandom number generator that generates uniformly distributed pseudorandom numbers using inverse transform sampling.

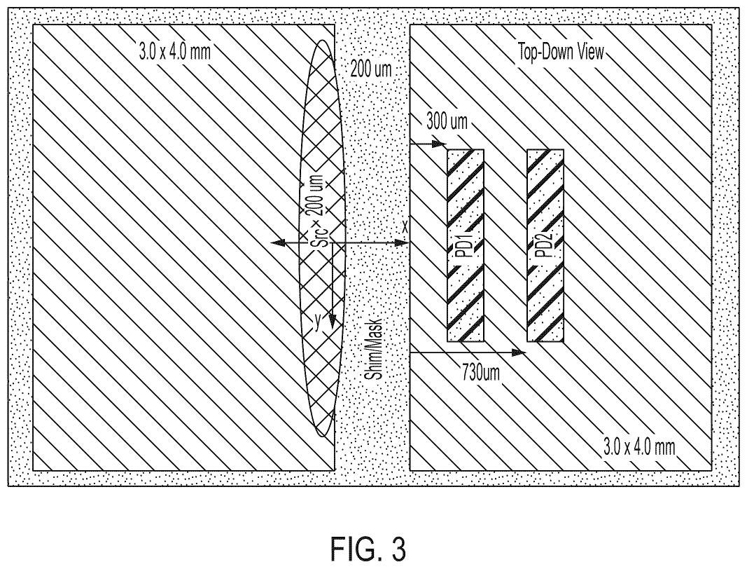

The geometry of the interface between the spectrophotometer and the tissue is shown in FIG. 3. The model of the geometry of the spectrophotometer and of the sample may be modeled, for the Monte Carlo simulations, using a set of module parameters, which may include (i) the radiance profile and alignment of the beam, (ii) the location and geometry of each photodetector, and (iii) the optical properties and thickness of the transmitting window and of the receiving window (which may be composed of glass). The output radiance profile may be modeled as a function of position on the surface of the transmitting window, angle, and wavelength, specifying the radiance (e.g., in units of power per unit solid angle per unit area). The tissue that is illuminated by the spectrophotometer and through which light propagates on its way back to the photodetector 112 may be modeled as consisting of several (e.g., two) layers, or “compartments”, with different properties, e.g., of an epidermis and a dermis. Within each layer, scattering of light may be modeled by a tissue scattering model, which may specify an attenuation length for scattering, Us. The attenuation length for scattering may be wavelength-dependent, and it may be different in the layers, e.g., it may have, at each wavelength, a first value in the dermis and a second value in the epidermis. If the scattering probability density function is an exponential probability density function, then the rate parameter of the exponential probability density function may be equal to the attenuation length for scattering, μₛ (e.g., the probability density function p(x) may be p(x)=e^{-μ}^{ₛ}^{x}).

Each Monte Carlo simulation may generate, as output, a collection of optical paths, each optical path being a sequence of rays related by scattering event, that, in the simulations, ended at the active surface of the photodetector. The optical paths may be grouped according to their length in each layer of the tissue scattering model. For example, in a two-layer tissue scattering model, a two-dimensional histogram may be formed based on (i) total path length in the first layer and (ii) total path length in the second layer. This histogram may be represented by a set of weights, including one weight for each bin of the histogram, each weight being proportional to the number of optical paths, from the simulation, that falls into the bin. In a two-layer tissue scattering model, each bin may correspond to (i) a range of values of the total path length in the first layer and (ii) a range of values of the total path length in the second layer. These results may equivalently be represented by a set of tuples {lⱼ, w}_{λ}, where, in a two-layer tissue scattering model, l₁ is a mid-range value identifying one of the ranges of values of the total path length in the first layer, l₂ is a mid-range value identifying one of the ranges of values of the total path length in the second layer, and w is proportional to the number of optical paths, in the Monte Carlo simulation, that fall into the bin identified by l₁ and l₂. The distribution of optical paths traversed in the Monte Carlo simulations (e.g., the distribution represented by the set of tuples {lⱼ, w}_{λ}) may be referred to as an optical path distribution. The weights w may represent both the (i) the proportion of the optical paths that extend various total distances within the different tissue compartments and (ii) the fraction of the output light that eventually scatters back to the photodetector 112.

The attenuation in each bin may then be calculated using a linear mixture model according to which

μ_{a,j}=Σᵢf_{i,j}μ_{a,i},
where f_{i,j} is the fraction or concentration of the pure component i (e.g., of the pure biomarker) in tissue compartment j, μ_{a,i} is the absorption coefficient of pure component i, and μ_{a,j} is the absorption coefficient for tissue compartment j.

As such, a baseline absorbance spectrum and an optical path distribution may be calculated from (i) as set of module parameters, and (ii) a set of tissue physiology parameters. The “baseline absorbance spectrum” may be calculated for nominal values of the concentration of any biomarker to be estimated. The tissue parameters may include a tissue scattering model, the thickness of each tissue compartment, and the concentrations or proportions of the constituents (e.g., water, collagen, and elastin) of the tissues of the subject. The baseline absorbance spectrum may then be calculated by (i) using a Monte Carlo simulation to calculate the distribution of optical paths light will take through the tissue, and (ii) using the linear mixture model to calculate the absorbance for each bin into which the optical paths are grouped.

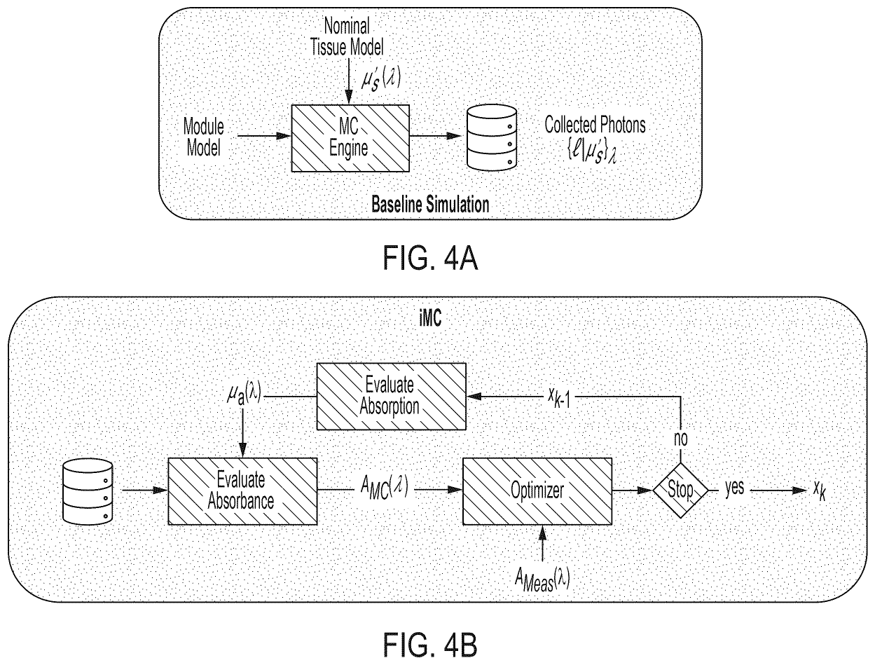

The parameters used for the baseline absorbance spectrum may be calculated using an inverse Monte Carlo method, as illustrated in FIGS. 4A and 4B. In such an approach, a nominal absorbance spectrum for a subject may be obtained using a baseline simulation, as shown in FIG. 4A (e.g., a single absorbance spectrum may be taken at an arbitrary time or a sequence of absorbance spectra may be taken over period of time and averaged together, or an absorbance spectrum may be taken and used as both a nominal absorbance spectrum and a test absorbance spectrum (discussed in further detail below)). As shown in FIG. 4B, tissue physiology parameters may then be found that, when used in a Monte Carlo simulation, produce an absorbance spectrum similar to the nominal absorbance spectrum for the subject. The search for such tissue physiology parameters may be performed iteratively, e.g., using (i) a gradient descent method in which a new Monte Carlo simulation is performed for each set of tentative tissue physiology parameters being tested, or (ii) a library of sets of tissue physiology parameters and the corresponding absorbance spectra, previously generated using Monte Carlo simulations. In FIG. 4B, A_{MC} is the absorbance spectrum calculated using the Monte Carlo simulation, and A_{Meas} is the measured absorbance spectrum. In the loop illustrated in FIG. 4B, the concentrations xₖ of the constituents (e.g., water, collagen, and elastin) of the tissue modeled in the Monte Carlo simulation are iteratively adjusted until a good match between A_{MC} and A_{Meas} is achieved.

The output radiance profile used in a Monte Carlo simulation may be calculated from the design geometry of the spectrophotometer, or measured, using one or more examples of spectrophotometers, or measured for a particular spectrophotometer, or it may be determined, along with other module parameters, using an inverse Monte Carlo method.

Once the tissue physiology parameters or the module parameters for a subject's module, or both, are known, a set of absorbance spectra may be calculated, for various hypothetical values of the biomarker of interest, using the optical path distribution, the absorption coefficient (as a function of wavelength) of the biomarker of interest, and the linear mixture model. These absorbance spectra may be referred to herein as “discriminant” absorbance spectra The concentration of the biomarker of interest in the tissue of the subject may then be estimated by (i) obtaining a test absorbance spectrum (an absorbance spectrum to be used to estimate the concentration of the biomarker of interest), and determining which of the discriminant absorbance spectra fits the test absorbance spectrum best. In determining how well a discriminant absorbance spectrum fits the test absorbance spectrum, wavelengths at which the signal-to-noise ratio of the test absorbance spectrum is high (e.g., at which the absorbance spectrum is highly sensitive to the presence of the biomarker of interest), and at which noise (e.g., due to absorbance fluctuations caused by unknown or uncontrolled fluctuations in the concentrations of other components of the subject's tissue) is small, may be weighted more heavily.

For example, an estimate of the concentration of a biomarker (e.g., a compound such as glucose, creatinine, urea, lactate, or alcohol within the tissues of the subject) may be generated by calculating the dot product of a measured absorbance spectrum with a weight vector (referred to herein as a b-vector) corresponding to the biomarker. Molecular information about the analyte is encoded in the g-vector (a distinct quantity from the g parameter of the Henyey-Greenstein function mentioned above), while noise covariance of the sample and instrument is calculated separately and combined to create a b-vector. At a wavelength at which the biomarker has a relatively large effect on the absorbance spectrum, the b-vector may have a relatively large value, and at a wavelength at which the biomarker has a relatively small effect on the absorbance spectrum, the b-vector may have a relatively small value. The magnitude of the effect on the absorbance spectrum of the biomarker may depend on the concentrations and absorbances of other components of the subject's tissues.

The b-vector may be calculated as follows:

$$b = \frac{{[ {X}_{n}^{T} {X}_{n} ]}^{- 1} g}{{{g}^{T} [ {X}_{n}^{T} {X}_{n} ]}^{- 1} g}$$

where Xₙ is the absorbance noise (e.g., the portion of the signal that is not correlated with biomarker concentration), and g is the product of (i) the spectral absorbance of the pure biomarker and (ii) the effective path (discussed in further detail below) through the tissues of the patient for the biomarker. The concentration of the biomarker may then be calculated as

$$y = X \cdot b$$

where X is either (i) a vector corresponding to a measured absorbance spectrum, or (ii) a two-dimensional array including a set of absorbance spectra taken at different times, and y is either (i) a calculated biomarker concentration (e.g., glucose, in mg/dL) or (ii) a set of calculated concentrations of the biomarker, each calculated for a time at which a respective absorbance spectrum was taken.

The effective path may be a measure of the length of the portion of each optical path that is within tissue components in which the biomarker is soluble. For example, if a biomarker is water-soluble, the concentration of the biomarker in water-containing components of the subject's tissues (e.g., in interstitial fluid and in blood) may be of greater interest than the average concentration of biomarker in the tissues sampled by the spectrophotometer. As such, for a water-soluble biomarker (e.g., glucose) the effective path may be the “water path” which may be a weighted average of length in water of each of the optical paths of the Monte Carlo simulation. Similarly, for other biomarkers, the effective path may be a weighted average, over all of the optical paths in the Monte Carlo simulation, of length in tissue components in which the biomarker is soluble.

The b-vector used to estimate the concentration of a biomarker may be (i) general (e.g., a b-vector that is calculated for an average subject and an average (or nominal) spectrophotometer) or (ii) subject-specific (e.g., a b-vector that is calculated for the tissue physiology parameters of the subject) or (iii) device specific (e.g., a b-vector that is calculated based on the module parameters of a particular spectrophotometer, or (iv) both subject-specific and device specific. The processing steps disclosed herein (e.g., the performing of Monte Carlo simulations, and the inferring of the concentration of a biomarker of interest) may be performed by one or more processing circuits. Each of the processing circuits may be, for example, (i) in a module (e.g., a wearable module) that includes the spectrophotometer or (ii) in another user device (e.g., a mobile telephone, a tablet, or a laptop computer), or (iii) in a cloud-based server.

Once the concentration of the biomarker has been estimated, the estimate may be transmitted, e.g., to a display (to display to a user (e.g., to the subject or to a clinician) the estimated concentration, or a status indicator (e.g., for glucose, “normal”, “dangerously low”, or “dangerously high”)) or it may be used to cause (e.g., automatically, under the control of a processing circuit) an intervention (e.g., (i) a command to an insulin pump to increase or decrease a flow rate of insulin in response to a high or low glucose concentration in the tissue of the subject, or (ii) disabling a vehicle if the concentration of alcohol (e.g., ethanol) in the subject's tissue is sufficiently high to indicate that the subject is likely to be impaired).

Some embodiments provide an improved ability to estimate concentrations of biomarkers in a subject's tissue, using a non-invasive method. As such, such embodiments provide an improvement to the technology of biomarker detection and of the estimating of concentrations of biomarkers in the tissue of a subject.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

Although exemplary embodiments of systems and methods for operating a spectrophotometer have been specifically described and illustrated herein, many modifications and variations will be apparent to those skilled in the art. Accordingly, it is to be understood that systems and methods for operating a spectrophotometer constructed according to principles of this disclosure may be embodied other than as specifically described herein. The invention is also defined in the following claims, and equivalents thereof.

What is claimed is:

1. A method, comprising:
estimating the concentration of a biomarker in a tissue sample, the estimating comprising:
performing a first simulation to calculate an optical path distribution for a tissue model;
obtaining, with a spectrophotometer, a first absorbance spectrum based on the tissue sample; and
estimating the concentration of the biomarker based on the optical paths and on the first absorbance spectrum.

2. The method of claim 1, comprising performing a first plurality of simulations, including the first simulation, to calculate the optical path distribution.

3. The method of claim 2, wherein each of the simulations is a Monte-Carlo simulation.

4. The method of claim 2, further comprising:
calculating an absorbance spectrum for each of the simulations;
measuring a second absorbance spectrum of the tissue sample; and
based on a similarity between:
an absorbance spectrum for a second simulation of the simulations and the second absorbance spectrum,
estimating a plurality of tissue physiology parameters of the tissue sample.

5. The method of claim 4, further comprising calculating a weight vector based on the tissue physiology parameters and on an absorption coefficient of the biomarker,
wherein the estimating of the concentration of the biomarker comprises calculating a dot product of the weight vector and the first absorbance spectrum.

6. The method of claim 4, further comprising:
calculating an absorbance spectrum for each of the simulations;
measuring a second absorbance spectrum of the tissue sample; and
based on the similarity between:
the absorbance spectrum for the second simulation and
the second absorbance spectrum,
estimating one or more parameters of the spectrophotometer.

7. The method of claim 4, further comprising calculating a weight vector based on the parameters of the spectrophotometer and on an absorption coefficient of the biomarker,
wherein the estimating of the concentration of the biomarker comprises calculating a dot product of the weight vector and the first absorbance spectrum.

8. The method of claim 7, further comprising:
based on a similarity between:
an absorbance spectrum for a second simulation of the simulations and the second absorbance spectrum,
estimating a plurality of tissue physiology parameters of the tissue sample.

9. The method of claim 8, further comprising calculating a weight vector based:
on the tissue physiology parameters,
on parameters of the spectrophotometer, and
on an absorption coefficient of the biomarker,
wherein the estimating of the concentration of the biomarker comprises calculating a dot product of the weight vector and the first absorbance spectrum.
